(19) United States
(12) Patent Application Publication
(10) Pub. No.: US 2013/0172785 A1
(43) Pub. Date: Jul. 4, 2013

(54) APPARATUS AND METHOD OF MEASURING FORCE OF EJACULATION

(71) Applicant: Jeffrey Lee Rosenblum, Chester Springs, PA (US)

(72) Inventor: Jeffrey Lee Rosenblum, Chester Springs, PA (US)

(21) Appl. No.: 13/340,347

(22) Filed: Dec. 29, 2011

(51) Int. Cl. A61B 5/103

(52) U.S. Cl. 600/587

(57) ABSTRACT

An apparatus for measuring the force of ejaculation including a cylinder having a length and an internal diameter sufficient to enclose an erect male penis, a groove track extending along at least part of the length of the cylinder, a membrane positioned within the cylinder and oriented substantially perpendicularly to the length of the cylinder, and a connector slide connected to the membrane, the connector slide being configured to suspend the membrane within the cylinder and to slide along groove track as the membrane is caused to moved lengthwise within the cylinder. A method of evaluating the force of ejaculation using the apparatus including inserting a penis into the cylinder and measuring the distance the membrane is propelled lengthwise within the cylinder by the force of semen ejected from the penis.

### CROSS-REFERENCE TO RELATED APPLICATIONS

This application claims the priority of U.S. Provisional Application No. 61/429,320, filed on Jan. 3, 2011, which is incorporated by reference herein in its entirety.

### FIELD OF INVENTION

The present invention relates to apparatus and method to assist in the evaluation of ejaculatory function and dysfunction, particularly but not exclusively for objectively measuring the force of ejaculation in a consistent manner.

### BACKGROUND

In the discussion of the background that follows, reference is made to certain structures and/or methods. However, the following references should not be construed as an admission that these structures and/or methods constitute prior art. Applicant expressly reserves the right to demonstrate that such structures and/or methods do not qualify as prior art.

Ejaculation is the integrated process of emitting semen from the penis, usually at a time concomitant with orgasm. Ejaculatory dysfunction is defined as a subjective lack of normal ejaculation. Ejaculatory dysfunction is part of the systemic impairment of sexual dysfunction. Ejaculatory dysfunction is commonly classified as premature ejaculation, delayed ejaculation, retrograde ejaculation, anejaculation, and painful ejaculation.

Determining the force of ejaculation is important in order to diagnose or quantify various types of ejaculatory dysfunction. Additionally, force of ejaculation is an important measurement to assess recovery after treatment or resolution of ejaculatory dysfunction. Furthermore, a number of surgical procedures (e.g., retroperitoneal lymph node dissection, colon resection), disease entities (i.e., diabetes, spinal cord injury), and medications (i.e., drugs for enlarged prostate and hypertension) can affect ejaculation.

The process of ejaculation can be assessed objectively by measuring the intravaginal latency time, volume of ejaculate, force of ejaculation, and direction of ejaculation, and terminal velocity of ejaculation. Force of ejaculation has been assessed on a nominal scale of 0 (no ejaculation), 1 (overflow ejaculation), 2 (ejaculation below the waistline), and 3 ejaculation past the waistline). This rudimentary method for measuring the force of ejaculation has been used to grade this parameter for years. The scale, however, presents several flaws in measurement: penis size and the distance from penis to waistline vary greatly from individual to individual; force is not truly assessed because nebulous distances are being measured with the scale; and no absolute finite measurement is obtained that can be utilized across groups of patient cohorts.

It is an object of the present invention to provide apparatus and methods for providing a device that can objectively measure the force of ejaculation in men in a consistent and reproducible manner.

### SUMMARY

An embodiment of an apparatus for measuring the force of ejaculation of semen from the penis of a male human includes a cylinder having a length and an internal diameter sufficient to enclose an erect male penis, a groove track extending along at least part of the length of the cylinder, a membrane positioned within the cylinder and oriented substantially perpendicularly to the length of the cylinder, and a connector slide connected to the membrane, the connector slide being configured to suspend the membrane within the cylinder and to slide along groove track as the membrane is caused to moved lengthwise within the cylinder.

An embodiment of a method of evaluating the force of ejaculation in a male human uses an apparatus including a cylinder and a membrane positioned within the cylinder and oriented substantially perpendicularly to the length of the cylinder. The method includes inserting the erect penis of a male human into the cylinder and measuring the distance the membrane is propelled lengthwise within the cylinder by the force of semen ejected from the penis.

It is to be understood that both the foregoing general description and the following detailed description are exemplary and explanatory and are intended to provide further explanation of the invention as claimed.

### BRIEF DESCRIPTION OF THE DRAWINGS

The following detailed description can be read in connection with the accompanying drawings in which like numerals designate like elements and in which:

FIG. 1 is a top view schematic illustrating one embodiment of apparatus for measuring force of ejaculation.

FIG. 2 is a series of side view schematics illustrating aspects of an apparatus as in FIG. 1. FIG. 2A shows an apparatus having a clear housing; FIG. 2B shows an apparatus having a distance measurement scale; and FIG. 2C shows an apparatus having movable distance markers.

### DETAILED DESCRIPTION

An apparatus is described herein to measure the force of ejaculation of semen from the penis of a human male. Such an apparatus can be used to evaluate the force of ejaculation, which is an important parameter in diagnosing ejaculatory dysfunction and assessing recovery of ejaculatory function after treatment or resolution of ejaculatory dysfunction. By quantifying the force of ejaculation and comparing this measurement to historical measurements for a given patient, as well as the mean value for this measurement obtained historically from men not defined as experiencing ejaculatory dysfunction, an objective assessment of an individual's ejaculatory function can be made.

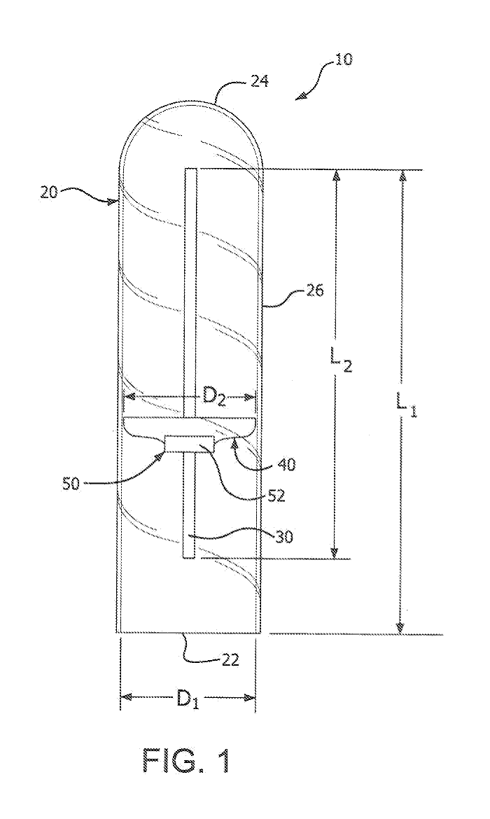

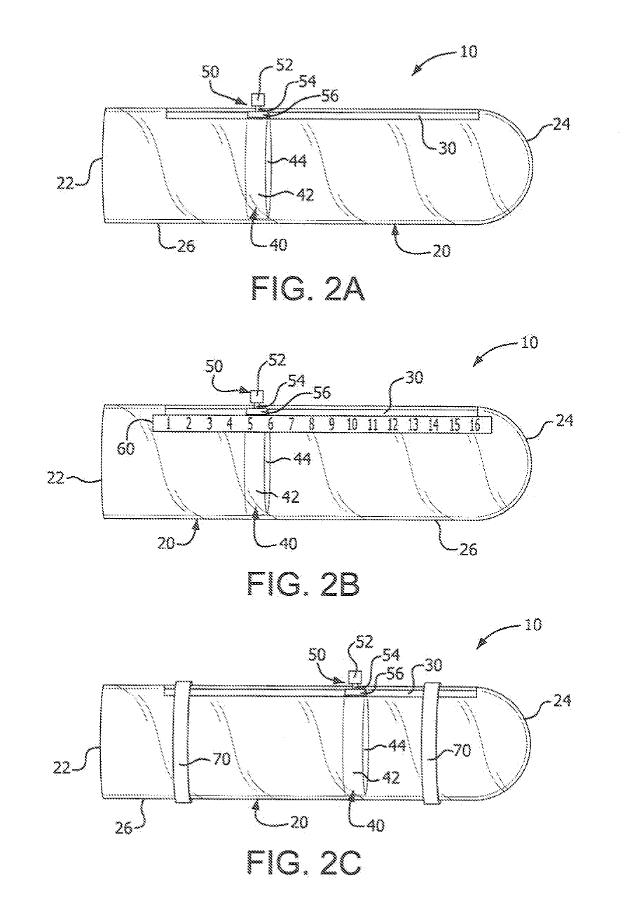

Examples of such an apparatus are shown in FIGS. 1 and 2. An exemplary apparatus 10 includes a cylinder 20 having a diameter at least as large as, and a length longer than, an erect male penis. A membrane 40 is positioned in the cylinder substantially perpendicularly with respect to the length of the cylinder 20. The membrane 40 is suspended by a connector slide 50 from a groove track 30 that runs along at least part of the length of the cylinder 20 so as to be substantially perpendicular to the length of the cylinder 20.

The apparatus 10 is able to quantify the force of ejaculation based on the distance the membrane 40 travels, when the membrane 40 is propelled along the length of the cylinder 20 by semen ejected from the penis. The distance measurement recorded for one man can be compared to distance measurements recorded for that man during previous ejaculatory events. Additionally, the distance measurement can be compared to the mean value for this measurement obtained historically from men not defined as experiencing ejaculatory dysfunction. Repeated use of the apparatus and repeated measurements can be made to ensure reproducibility.

In the depicted embodiments, the cylinder 20 has a cylindrical wall 26, a first end 22, and a second end 24. The first end 22 is open to enable insertion of a penis. The second end 24 may be open or closed. The cylinder 20 is defined by a length L1 and a diameter D1. The diameter D1 of the cylinder 20 must be sufficiently large to accommodate an erect penis of a human. The length L1 of the cylinder 20 should be sufficiently long to enable measurement of the force of semen ejaculated from the penis using a movable membrane 40 as described herein.

The cylinder 20 is provided with the groove track 30, which may be formed integrally with the cylinder 20 as a longitudinal slot in the cylinder wall 26. Alternatively, the groove track 30 may be mounted to the inside of the cylinder wall 26, or to the outside of the cylinder wall 26 but made accessible via a slot or opening in the cylinder wall 26. The groove track 30 extends along at least a part of the length of the cylinder 20, terminating at or near the second end 24 of the cylinder 20.

The membrane 40 is configured to be movable, with minimal frictional resistance, along the length of the cylinder 20. The membrane 40 may include a stiff circular rim 42 bonding a lightweight membrane member 44 held generally taught by the rim 42. The membrane 40 has an outer diameter D2 that is equal to or slightly less than the internal diameter D1 of the cylinder 20.

The connector slide 50 is provided to secure the membrane 40 to the cylinder 20, to guide the membrane 40 in sliding along the length of the cylinder 20, and to enable measurement of the movement of the membrane 40 in response to ejaculation from a penis. In the depicted embodiment, the connector slide 50 is generally T-shaped and includes a neck portion 54 sized to slide within the groove track 30, a head portion 52 sized larger than the groove track 30 located at one end of the neck portion 54, and a base portion 56 connected to the membrane 40 at the other end of the neck portion 54. The connector side 50 suspends the membrane 40 within the cylinder 20 so that the membrane 40 remains substantially perpendicular to the length of the cylinder 20 as the membrane 40 slides within the cylinder and the connector slide 50 slides within the groove track 30.

In one variation, as shown in FIG. 2A, the wall 26 of the cylinder 20 may be made at least partially from a clear material to enable viewing of the location of the membrane 40 within the cylinder 20. In another variation, as shown in FIG. 2B, a measurement scale 60 is provided along the groove track 30 to measure the travel distance of the membrane 40. In another variation, as shown in FIG. 2C, one or more markers 70 are provided that can be moved along the length of the cylinder 20 to indicate a starting and stopping distance of the membrane 40, i.e., before and after ejaculation, respectively.

In use, the penis of a male human (erect if possible) is inserted into the open end 22 of the cylinder 20 of the apparatus 10. Preferably this is done just prior to ejaculation. The apparatus 10 may be held level or tilted slightly so that the tip of the penis contacts the membrane 40 and positions the membrane 40 and the connector slide 50 at a starting position along the groove track 30. The starting position of connector slide 50 the should be recorded once penis is in position prior to ejaculation, for example with reference to the measurement scale 60 or by locating one of the markers 70 at the starting position.

When semen is ejaculated from the penis, the force of ejaculation propels the membrane 40, guided by the connector slide 50 on the groove track 30, to a stopping position. The higher the force of ejaculation, the farther the membrane 40 and connector slide 50 will be propelled along the groove track 30. The stopping position of connector slide 50 along the groove track should be recorded.

force of ejaculation can be calculated based on the distance between the stopping position and starting position of the connector slide 50 along the track groove 30, and applying an appropriate scaling constant to translate that distance into force. The scaling constant may be based on several factors, including but not limited to the frictional force between the membrane 40 and the cylinder wall 26, the frictional force between the slide connector 50 and the groove track 30, and the angle at which the apparatus is held during use.

The invention provides a method and apparatus for evaluation of ejaculatory function, diagnosis of ejaculatory dysfunction, and assessment of recovery from treatment of ejaculatory dysfunction. Although described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. An apparatus for measuring the force of ejaculation of semen from the penis of a male human comprising:
a cylinder having a length and an internal diameter sufficient to enclose an erect male penis;
a groove track extending along at least part of the length of the cylinder;
a membrane positioned within the cylinder and oriented substantially perpendicularly to the length of the cylinder; and
a connector slide connected to the membrane, the connector slide being configured to suspend the membrane within the cylinder and to slide along groove track as the membrane is caused to moved lengthwise within the cylinder.

2. The apparatus of claim 1, wherein said tube is of sufficient length both to enclose the penis and to allow sufficient room distal to the penis for movement of the membrane in response to ejaculation from the penis.

3. The apparatus of claim 1, wherein at least a portion of the cylinder is clear to enable viewing of the movement of the membrane.

4. The apparatus of claim 1, further comprising a measurement scale along the track groove for measuring the distance the membrane is moved lengthwise within the cylinder in response to ejaculation from the penis.

5. The apparatus of claim 1, further comprising one or more movable markers positionable along the length of the cylinder for marking locations of the membrane as the membrane is moved lengthwise within the cylinder in response to ejaculation from the penis

6. A method of evaluating the force of ejaculation in a male human using an apparatus including a cylinder and a membrane positioned within the cylinder and oriented substantially perpendicularly to the length of the cylinder, the method comprising:
inserting the erect penis of a male human into the cylinder;
causing the male human to ejaculate , and measuring the distance the membrane is propelled lengthwise within the cylinder by the force of semen ejected from the penis.

7. The method of claim 6, further comprising:
calculating a force of ejaculation based on the distance of movement of the membrane; and
comparing the calculated force of ejaculation to known standard measurements for evaluating ejaculation.
